(19) United States
(12) Patent Application Publication
(10) Pub. No.: US 2004/0267375 A1
(43) Pub. Date: Dec. 30, 2004

(54) Hip joint endoprosthesis

(76) Inventor: Arno Friedrichs, Kulmbach (DE)

Correspondence Address: WILLIAM COLLARD, COLLARD & ROE, P.C., 1077 NORTHERN BOULEVARD, ROSLYN, NY, 11576, US

(21) Appl. No.: 10/874,059

(22) Filed: Jun. 22, 2004

(30) Foreign Application Priority Data
Jun. 27, 2003 (DE) 103 28 949.6

(51) Int. Cl. A61F 2/32

(52) U.S. Cl. 623/22.18; 623/23.41

(57) ABSTRACT

A hip joint endoprosthesis comprises a shank, a joint ball at the upper end region of the shank, a ball socket, within which the joint ball is mounted to be rotationally movable, and a joint gap present between the joint ball and the joint socket. A liquid-filled or gas-filled rolling-contact bag is provided in the joint gap.

### BACKGROUND OF THE INVENTION

1. Field of the Invention

The invention relates to a hip joint endoprosthesis which comprises a shank, a joint ball at the upper end region of the shank, a joint socket, within which the joint ball is mounted to be rotationally movable, and a joint gap between the joint ball and the joint socket.

2. The Prior Art

Hip joint endoprostheses of that kind are already known. They are used as a substitute for degenerated natural hip joints in the human body. For insertion of the shank with the joint ball provided at the upper end region thereof, the upper end of the femur is initially cleanly sawn off. Insertion of the downwardly tapering shank into the marrow channel with or without use of bone cement is then carried out. For insertion of the joint socket, the natural joint socket of the human body is precisely routed away and then the artificial joint socket is inserted into the routed-out region. The joint socket is fixed by a thread on its outer region, which is screwed into the routed-out natural joint socket. Alternatively thereto, the joint socket can also be cemented in place in the routed-out natural joint socket and in a given case additionally fixed by means of screws.

Known hip joint endoprosthesis generally consist of metallic materials, ceramic materials, plastics materials or a mix of metal and ceramic.

A problem of known artificial hip joints consists in that due to the loads, which occur in daily life, in the joint, friction and wear arise which with time can lead to undesired secondary effects such as notch fissures in the joint ball or the joint socket or even to detaching of the shank from the femur. These undesired effects occur to increased extent with production inaccuracies. The consequences are a reduced service life of the hip joint endoprosthesis.

### SUMMARY OF THE INVENTION

It is therefore an object of the invention to provide a hip joint endoprosthesis in which the frequency of undesired secondary effects is reduced.

This object is met by a hip joint endoprosthesis comprising a shank, a joint ball at the upper end region of the shank, a ball socket, within which the joint ball is mounted to be rotationally movable, and a joint gap present between the joint ball and the joint socket. A liquid-filled or gas-filled rolling-contact bag is provided in the joint gap.

The advantages of the invention consist particularly in that due to the use of a liquid-filled or gas-filled rolling-contact bag provided in the joint gap, rubbing of the joint ball and joint socket against one another in the case of a relative movement, which takes place under load, between the joint ball and the joint socket is prevented. Rather, there takes place a rolling movement in which the joint ball and the joint socket in the ideal case do not even come into contact with one another, since the interposed liquid-filled or gas-filled rolling-contact bag acts as a buffer. By comparison with known hip joint endoprostheses, reduced loadings of the mutually facing surfaces of the joint ball and joint socket thereby occur. Less friction and less wear occur, which has the consequence of a reduced number of notch fissures in the material. In addition, the probability of detaching the shank from the femur is reduced. The consequence is an extended service life of the hip joint endoprosthesis.

If the liquid-filled or gas-filled rolling-contact bag is fixedly connected at a fixing point with the joint ball or the joint socket, then it is ensured that it cannot slip in the case of a relative movement between joint ball and joint socket.

Preferably there is provided in the region of the joint gap a sealing element, particularly a sealing ring, which prevents clamping in place of the liquid-filled or gas-filled rolling-contact bag or clamping in place of substances, which belong to the body, in the joint gap.

### BRIEF DESCRIPTION OF THE DRAWINGS

Other objects and features of the present invention will become apparent from the following detailed description considered in connection with the accompanying drawings. It is to be understood, however, that the drawings are designed as an illustration only and not as a definition of the limits of the invention.

In the drawings, wherein similar reference characters denote similar elements throughout the several views:

FIG. 1 shows a sketch of a sectional illustration of a hip joint endoprosthesis according to a first embodiment of the invention;

FIG. 2 shows a sketch of a sectional illustration of a hip joint endoprosthesis according to a second embodiment of the invention;

FIG. 3 shows a sketch of a sectional illustration of a hip joint endoprosthesis according to a third embodiment of the invention; and

FIG. 4 shows a sketch of a sectional illustration of a hip joint endoprosthesis according to a fourth embodiment of the invention.

### DETAILED DESCRIPTION OF THE PREFERRED EMBODIMENT

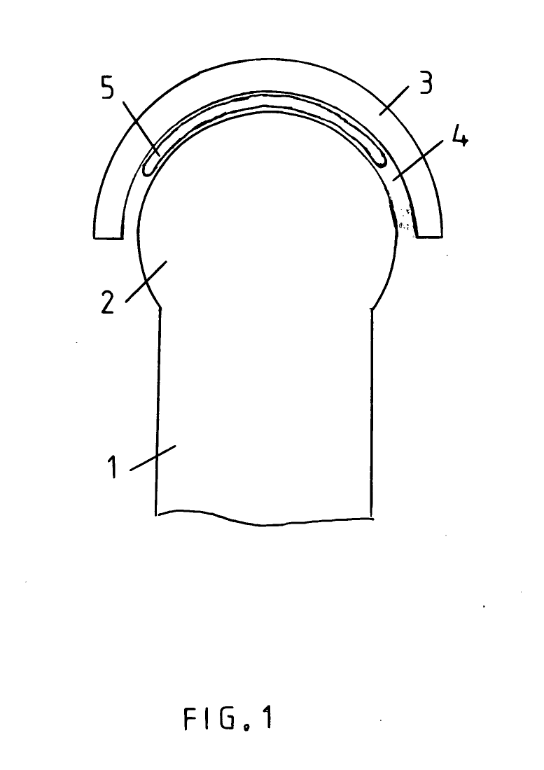

Referring now in detail to the drawings, FIG. 1 shows a sketch of a sectional illustration of a hip joint endoprosthesis according to a first embodiment of the invention. The illustrated hip joint endoprosthesis comprises a shank 1 which is shown cut away and at the upper end region of which is provided a joint ball 2. This joint ball is either a separate component which is placed on the shank or a component integrally formed with the shank.

In addition, the illustrated hip joint endoprosthesis comprises a joint socket 3, within which joint ball 2 is mounted to be rotationally movable. A joint gap 4 is present between joint ball 2 and joint socket 3.

A liquid-filled rolling-contact bag 5 is provided in joint gap 4. If a relative movement between joint ball 2 and joint socket 3 arises under load, then during this movement the joint ball and joint socket do not rub directly against one another, but there takes place a rolling movement in which the liquid-filled rolling-contact bag serves as a buffer. Due to avoidance of a direct rubbing of joint ball and joint socket against one another, the surfaces of the joint ball and joint socket are subjected to a reduced loading. The probability of formation of abrasion particles and creation of notch fissures in the material is thereby reduced by comparison with known hip joint endoprostheses. This has the consequence of a longer service life of a hip joint endoprosthesis according to the invention.

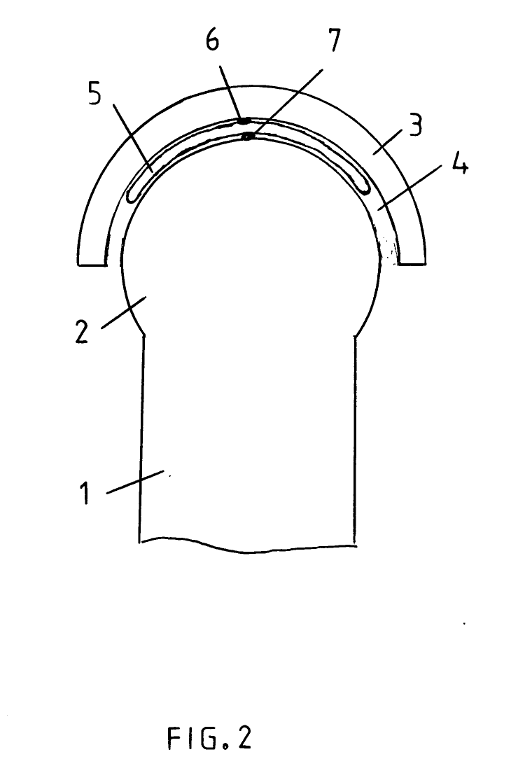

FIG. 2 shows a sketch of a sectional illustration of a hip joint endoprosthesis according to a second embodiment of the invention. The illustrated hip joint endoprosthesis comprises a shank 1 which is illustrated cut away and at the upper end region of which there is provided a joint ball 2. This joint ball is either a separate component placed on the shank or a component integrally formed with the shank.

In addition, the illustrated hip joint endoprosthesis comprises a joint socket 3, within which joint ball 2 is mounted to be rotationally movable. A joint gap 4 is present between joint ball 2 and joint socket 3.

A liquid-filled rolling-contact bag 5 is provided in joint gap 4. If a relative movement between joint ball 2 and joint socket 3 arises under load, then during this movement the joint ball and joint socket do not rub directly against one another, but there takes place a rolling movement in which the liquid-filled rolling-contact bag serves as a buffer. By virtue of avoidance of a direct rubbing of the joint ball and joint socket against one another, the surfaces of the joint ball and joint socket are subjected to a reduced loading. The probability of formation of abrasion particles and creation of notch fissures in the material is thereby reduced by comparison with known hip joint endoprostheses. This has the consequence of a longer service life of a hip joint endoprosthesis provided with the features according to the invention.

In order to prevent slipping of the liquid-filled rolling-contact bag 5 within joint gap 4, rolling-contact bag 5 is fixedly connected at a fixing 6 with joint socket 3. This fixed connection can be achieved, for example, in that not only joint socket 3, but also rolling-contact bag 5 consist of a plastics material and the two plastics materials are glued together in the contact region or are connected together by a suitable mechanically positive coupling. Alternatively or additionally there can be used, instead of fixing point 6, also a fixing point 7 which is provided at joint ball 2.

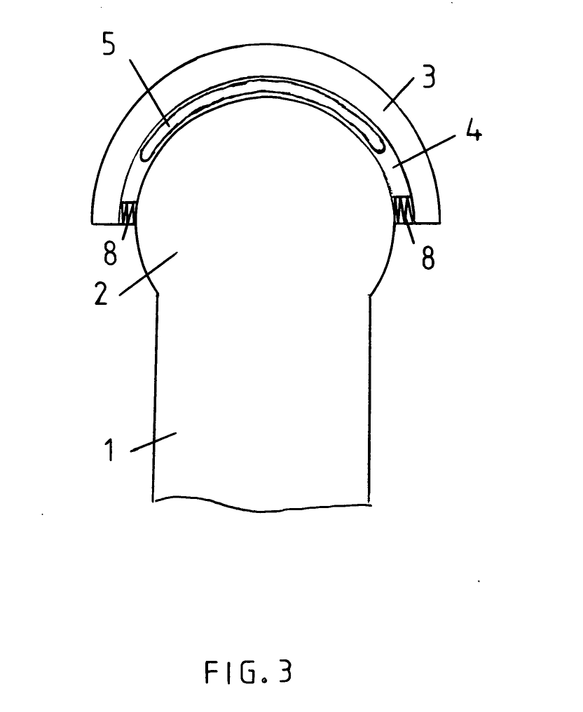

FIG. 3 shows a sketch of a sectional illustration of a hip joint endoprosthesis according to a third embodiment of the invention. The illustrated hip joint endoprosthesis comprises a shank 1 which is shown cut away and at the upper end region of which is provided a joint ball 2. Joint ball 2 is either a separate component placed on the shank or a component formed integrally with the shank.

In addition, the illustrated hip joint endoprosthesis comprises a joint socket 3, within which joint ball 2 is mounted to be rotationally movable. A joint gap 4 is present between joint ball 2 and joint socket 3.

A liquid-filled rolling-contact bag 5 is provided in this joint gap 4. If a relative movement between joint ball 2 and joint socket 3 occurs under load, then joint ball 2 and joint socket 3 do not rub directly against one another during this movement, but there takes place a rolling movement in which the liquid-filled rolling-contact bag serves as a buffer. By avoiding a direct rubbing of the joint ball and joint socket against one another, the surfaces of the joint ball and the joint socket are subjected to a reduced loading. The probability of formation of abrasion particles and creation of notch fissures in the material is thereby reduced by comparison with known hip joint endoprostheses. This has the consequence of a longer service life of a hip joint endoprosthesis provided with the features according to the invention.

In order to avoid, particularly in the case of large relative movements between the joint ball and joint socket, a clamping in place of the liquid-filled rolling-contact bag between the inner edge of the lower rim of the joint socket and the joint ball the hip joint endoprosthesis illustrated in FIG. 2 has a sealing element 8 provided in joint gap 4. This is preferably a sealing ring which is fastened to joint socket 3 and surrounds the entire joint ball 2. This sealing element additionally prevents, in advantageous manner, organic material of the human body, which is present in the vicinity of the joint gap, from being pinched in joint gap 4 on occurrence of relative movements between the joint ball and the joint socket and possibly even being able to be nipped off.

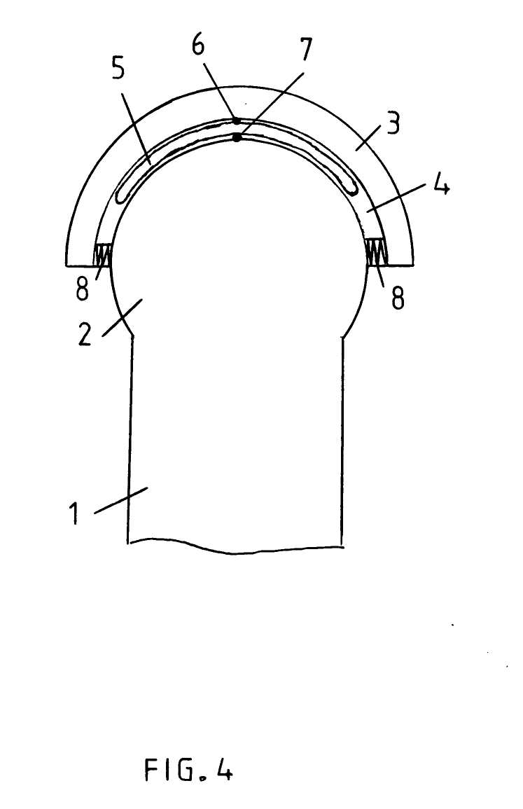

FIG. 4 shows a sketch of a sectional illustration of a hip joint endoprosthesis according to a fourth embodiment of the invention. This illustrated hip joint endoprosthesis comprises a shank 1 which is shown cut away and at the upper end region of which there is provided a joint ball 2. This joint ball is either a separate component placed on the shank or a component formed integrally with the shank.

In addition, the illustrated hip joint endoprosthesis comprises a joint socket 3, within which joint ball 2 is mounted to be rotationally movable. A joint gap 4 is present between joint ball 2 and joint socket 3.

A liquid-filled rolling-contact bag 5 is provided in joint gap 4. If a relative movement between joint ball 2 and joint socket 3 occurs under load then the joint ball and joint socket do not rub directly against one another during this movement, but there takes place a rolling movement in which the liquid-filled rolling-contact bag serves as a buffer. By avoiding a direct rubbing of the joint ball and joint socket against one another, the surfaces of the joint ball of the joint socket are exposed to reduced loading. The probability of formation of abrasion particles and creation of notch fissures in the material is thereby reduced by comparison with known hip joint endoprostheses. This has the consequence of a longer service life with a hip joint endoprosthesis provided with the features according to the invention.

In order to prevent slipping of liquid-filled rolling-contact bag 5 within joint gap 4, rolling-contact bag 5 is fixedly connected at a fixing point 6 with joint socket 3. This fixed connection can be achieved, for example, in that not only the joint socket, but also the rolling-contact bag consist of a plastics material and the two plastics materials are glued together in the contact region or are connected together by a suitable mechanically positive coupling. Alternatively or additionally thereto there can be used, instead of fixing point 6, also a fixing point 7 which is provided at joint ball 2.

In order to prevent pinching or even nipping off of organic material of the human body which is present in the vicinity of the joint gap, due to relative movements between the joint ball and the joint socket, the hip joint endoprosthesis illustrated in FIG. 4 has a sealing element 8 provided in joint gap 4. This is preferably a sealing ring which is fastened to joint socket 3 and surrounds entire joint ball 2.

The above-described embodiments of the invention have described the rolling-contact bag as being filled with liquid. Alternatively, it is also possible to fill the rolling-contact bag with a gas.

Rolling-contact bag 5 is preferably made of a plastics material with high tear resistance and a high degree of resilience. The liquid itself can be, for example, water or oil. Oxygen, nitrogen or liquid gas can be used as gas.

Accordingly, while only a few embodiments of the present invention have been shown and described, it is obvious that many changes and modifications may be made thereunto without departing from the spirit and scope of the invention.

What is claimed is:

1. A hip joint endoprosthesis comprising:
a shank;
a joint ball disposed at an upper end region of said shank;
a joint socket, within which the joint ball is mounted to be rotationally movable;
a joint gap disposed between the joint ball and the joint socket; and
a liquid-filled or gas-filled rolling-contact bag disposed in the joint gap.

2. The hip joint endoprosthesis according to claim 1, wherein the rolling-contact bag is fixedly connected at a fixing point with the joint ball or the joint socket.

3. The hip joint endoprosthesis according to claim 1, further comprising a sealing element disposed in the joint gap.

4. The hip joint endoprosthesis according to claim 3, wherein the sealing element is a sealing ring.

5. The hip joint endoprosthesis according to claim 3, wherein the sealing element is fixedly connected with the joint socket.
